(19) United States
(12) Patent Application Publication
(10) Pub. No.: US 2018/0325391 A1
(43) Pub. Date: Nov. 15, 2018

(54) CABLE HUB APPARATUS

(71) Applicant: Edwards Lifesciences Corporation, Irvine, CA (US)

(72) Inventors: Brandon S. Weiler, Mission Viejo, CA (US); Ammar Manghi, Tustin, CA (US); Christopher Ikei, Irvine, CA (US); Wei-Jiun Liu, Newport Beach, CA (US); John A. Frazier, Costa Mesa, CA (US); Ramin Mousavi, Tustin, CA (US); Richard B. Paul, Yorba Linda, CA (US); Sandarsh Mohan Kumar, Irvine, CA (US); Shaun Fetherston, Coto de Caza, CA (US)

(21) Appl. No.: 15/776,754

(22) PCT Filed: Nov. 18, 2015
(86) PCT No.: PCT/US2015/061254

§ 371 Date: May 16, 2018

(51) Int. Cl. A61B 5/0205; A61B 5/0402

(52) U.S. Cl. CPC A61B 5/02055; A61B 5/0402; A61M 5/14; A61B 2562/227; A61B 2560/0431

(57) ABSTRACT

Embodiments of the invention disclosed herein relate generally to a cable hub apparatus. The cable hub apparatus is configured to communicate physiological data for a patient to a patient monitoring device. The cable hub apparatus includes: a plurality of analog sensor connectors configured to receive analog physiological sensor data for the patient; a plurality of digital sensor connectors configured to received digital physiological sensor data for the patient; a controller to control collecting the received physiological sensor data; and a digital interface configured to communicate through a single cable the received physiological sensor data to the patient monitoring device.

### CROSS-REFERENCE TO RELATED APPLICATIONS

This application claims the benefit of International Patent Application No. PCT/US2015/61254, filed Nov. 18, 2015, the contents of which are incorporated by reference herein in their entireties for all purposes.

### BACKGROUND

### Field

The present invention relates to a cable hub apparatus configured to communicate physiological data for a patient to a patient monitoring device.

### Relevant Background

With reference to FIG. 1A, an example of a medical environment is shown. FIG. 1A shows a known configuration 100 of medical apparatuses for physiological monitoring and IV administration in an operating room. A standalone IV pole 105 is provided. Secured to the IV pole 105 are a monitoring device 110, a connection accessory 115, and an IV bag 120. A power supply unit 125 connected to an immobile AC power supply is utilized to supply power to monitoring device 110 and the connection accessory 115. One or more sensor cables 130 are connected to physiological sensors attached to the patient on one end and to connection accessory 115 on the other. One or more cables connect monitoring device 110 to connection accessory 115 to allow transfer of sensor-related information. On the other side of the patient bed is an anesthesia machine 135. An additional cable 133 connects anesthesia machine 135 to connection accessory 115 to allow anesthesia machine 135 to make use of sensor-related information.

There are several drawbacks to this known configuration: as can be seen in FIG. 1A, there is a clutter of cables around connection accessory 115. Moreover, transporting the patient to a different location (e.g., from an Emergency Room (ER) to an intensive care unit (ICU)) is inconvenient because in addition to disconnecting and reconnecting cable 133 between connection accessory 115 and anesthesia machine 135, power supply unit 125 also needs to be disconnected before transportation and reconnected after transportation. Of course, even more cables may need to be disconnected and reconnected if monitoring device 110 is not transported together with the patient.

To address these drawbacks, embodiments of the invention described hereinafter utilize a streamlined configuration including a cable hub apparatus with patient cable connectors/interfaces.

### SUMMARY

Embodiments of the invention disclosed herein relate generally to a cable hub apparatus. The cable hub apparatus is configured to communicate physiological data for a patient to a patient monitoring device. The cable hub apparatus includes: a plurality of analog sensor connectors configured to receive analog physiological sensor data for the patient; a plurality of digital sensor connectors configured to received digital physiological sensor data for the patient; a controller to control collecting the received physiological sensor data; and a digital interface configured to communicate through a single cable the received physiological sensor data to the patient monitoring device.

### BRIEF DESCRIPTION OF THE DRAWINGS

FIG. 1A shows a known configuration of medical apparatuses for physiological monitoring and IV administration in an operating room.

FIG. 1B shows an improved configuration of medical apparatuses for physiological monitoring and IV administration in an operating room including a cable hub apparatus with patient cable connectors/interfaces, according to one embodiment of the invention.

FIG. 2A is perspective view of an example cable hub apparatus, according to one embodiment of the invention.

FIG. 2B is another perspective view of an example cable hub apparatus, according to one embodiment of the invention.

FIG. 3 is a diagram illustrating an exemplary hardware architecture for a cable hub apparatus, according to one embodiment of the invention.

### DETAILED DESCRIPTION

Embodiments of the invention generally relate to a system, patient monitoring devices, and a cable hub apparatus. In particular, the cable hub apparatus is configured to communicate physiological data for a patient to a patient monitoring device. The cable hub apparatus may comprise: a plurality of analog sensor connectors configured to receive analog physiological sensor data for the patient; a plurality of digital sensor connectors configured to receive digital physiological sensor data for the patient; a controller to control collecting the received physiological sensor data; and a digital interface configured to communicate through a single cable the received physiological sensor data to the patient monitoring device.

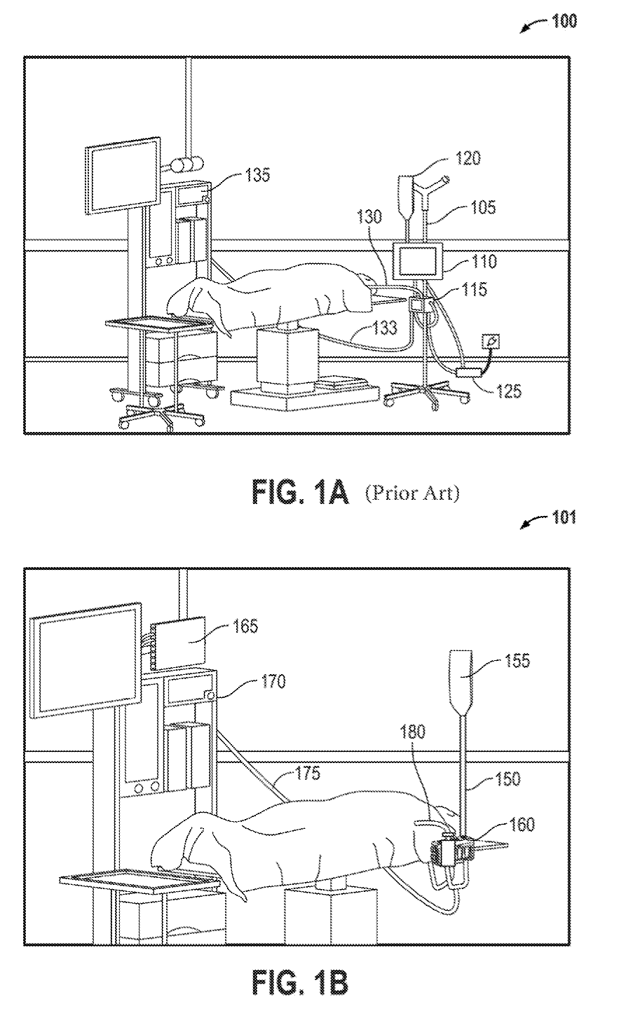

FIG. 1B shows an improved configuration 101 of medical apparatuses for physiological monitoring and IV administration in an operating room including a cable hub apparatus 160 with patient cable connectors/interfaces to be described hereinafter. An IV pole 150 may be securely fastened to or provided as a part of a patient bed. In some embodiments, IV pole 150 may be a standalone unit separate from the patient bed. An IV bag 155 may be attached IV pole 150. A cable hub apparatus 160 may be provided and secured to IP pole 150. One or more sensor cables 180 may be connected to physiological sensors (e.g., analog or digital physiological sensors) attached to the patient on one end and to cable hub apparatus 160 on the other. On the other side of the patient bed, a monitoring device 165 may be secured to an anesthesia machine 170. Monitoring device 165 may be connected to cable hub apparatus 160 via a single common interface cable 175 and supplies power to the cable hub apparatus 160 via the single common interface cable 175. In other words, common interface cable 175 may carry physiological sensor-related data and other data, as well as electric power. Further, in some embodiments, power may also be provided through the cable hub apparatus 160 to the physiological sensors. Monitoring device 165 may be connected to anesthesia machine 170 to pass on sensor-related information received from cable hub apparatus 160 so that anesthesia machine 170 may make use of the sensor-related information.

When a patient in the patient bed is to be transported, only common interface cable 175 needs to be disconnected. IV pole 150 and objects attached thereto including IV bag 155 and cable hub apparatus 160, as well as sensor cables 180, may be easily transported together with the patient. Because IV pole 150 is securely fastened to or provided as a part of the patient bed, no extra effort is required to move IV pole 150. Once the patient in the patient bed reaches the destination location (e.g., from the ER to the ICU), all the physiological sensors may resume working after a monitoring device 165 at the destination location is connected to cable hub apparatus 160 with a single common interface cable 175.

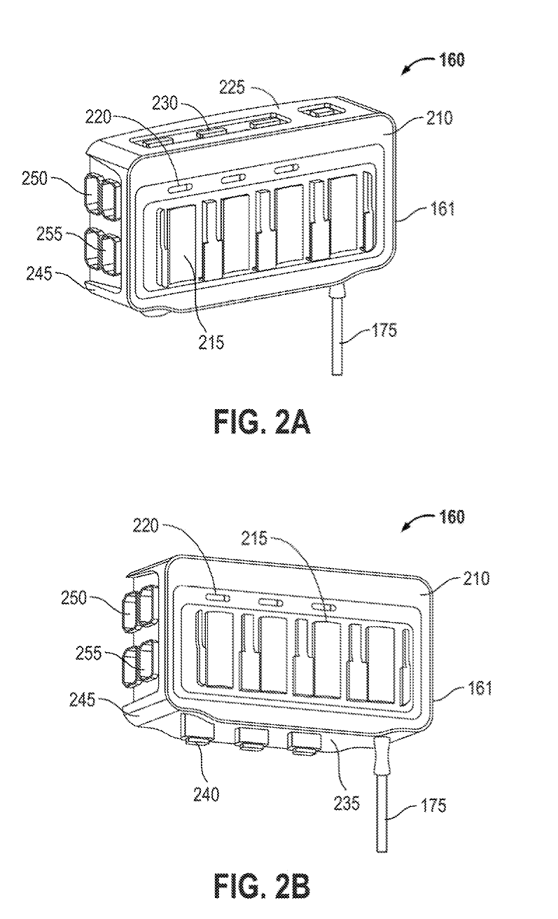

Referring to FIGS. 2A and 2B, illustrations of perspective views of an example cable hub apparatus 160 are shown. As can be seen, the external housing 161 of cable hub apparatus 160 may be approximately of a rectangular cuboid shape including six sides. On a first side 210 (front side shown in FIGS. 2A and 2B), of external housing 161, one or more sensor holders 215 may be provided. Sensors that may be placed into sensor holders 215 may include, for example, disposable pressure transducers (DPTs). In one example, four sensor holders 215 may be provided to hold four pressure sensors, such as DPTs. As an example, the four DPTs may be for the measuring of Pulmonary Artery Pressure (PAP), Central Venous Pressure (CVP), Arterial Pressure (AP), and for the use with a Venous Arterial blood Management Protection (VAMP) system, respectively. It should be appreciated that these are merely examples, and any type of mountable sensor may be utilized. Further, the sensor holders 215 may be removable. In addition to sensor holders 215, one or more LED indicators 220 for indicating the connection/operation conditions of one or more DPTs may be provided on first side 210. In one example, there may be three LED indicators 220 on first side 210, each corresponding to one DPT, as will be described in more detail below.

On a second side 225 (top side shown in FIGS. 2A and 2B), of external housing 161, one or more push buttons 230 may be provided. Push buttons 230 may be part of push button switches that control physiological sensor-related functionalities. For example, one of push buttons 230 may be a zeroing button that zeroes a connected DPT when depressed. Pressure zeroing is a function that some monitoring devices may utilize to zero the pressure signal from a pressure sensor device, such as a DPT, to the atmospheric pressure. Zeroing is typically performed prior to pressure measurement and is commonly repeated when the position of the pressure sensor device is changed. Another one of push buttons 230 may be an alarm silence button that silences an active alarm when depressed. When a push button 230 is depressed, a corresponding signal is transmitted from cable hub apparatus 160 to monitoring device 165 via single common interface cable 175. For example, signals related to zeroing and/or alarm silencing may be transmitted. In one example, as shown in FIG. 2A, four push buttons 230 may be provided on second side 225, among which three (e.g., leftmost) may be zeroing buttons for three DPTs, respectively, and a fourth button (e.g., rightmost) may be an alarm silence button.

On a third side 235 (bottom side shown in FIG. 2B), of external housing 161, one or more analog sensor connectors/interfaces 240 to which analog physiological sensors may be connected via a cable are be provided. In this way, an analog sensor connector/interface 240 may receive analog physiological sensor data for a patient from an analog physiological sensor coupled to the patient via a cable. An analog physiological sensor may be, for example, a pressure sensor such as a DPT. In one example, three analog sensor connectors 240 may be provided on third side 235. In one particular example, the three analog sensor connectors 240 may be three DPT interface connectors. For example, as previously described, DPTs may be mounted to the sensor holders 215 and connected via cables to the DPT connectors 240. Furthermore, cable hub apparatus 160 may be attached to a single common interface cable 175 through the third side 235 to the patient monitoring device 165.

For example, common interface cable 175 may be attached to cable hub apparatus 160. If common interface cable 175 is removably attached, a common interface connector for common interface cable 175 may be provided on third side 235. Further, the common interface cable/connector is electrically connected to a common interface of the hardware architecture of cable hub apparatus 160, as will be described in more detail below.

As will be more particularly described hereinafter, common interface cable 175 may carry sensor-related data for multiple sensors (e.g., both analog physiological sensors and digital physiological sensors) to the monitoring device 165, as well as, to supply power from the monitoring device 165 to the cable hub apparatus 160, and, in turn, to the physiological sensors connected to cable hub apparatus 160. Moreover, as will be described below, common interface cable 175 may also carry a heater signal that may be required by a physiological sensor that includes a heater.

On a fourth side 245 (left side shown in FIGS. 2A and 2B), of external housing 161, one or more digital sensor connectors/interfaces 250 (e.g., three digital sensor connectors/interfaces) to which digital physiological sensors that output digital signals may be connected via a cable may be provided. In this way, a digital sensor connector/interface 250 may receive digital physiological sensor data for a patient from a digital physiological sensor coupled to the patient via a cable. A physiological sensor that outputs digital signals may be, for example, a cardiac output sensor, an oximeter, a temperature sensor, etc.

It should be appreciated that the previously described physiological sensors may be analog or digital and may be invasive or non-invasive. Examples of sensor devices may include pressure sensors, temperature sensors, image sensors, light sensors, electric sensors, magnetic sensors, flow sensors, biosensors, accelerometer sensors, etc., that may be used to measure patient physiological data such as: cardiac measurements, blood measurements, chemical measurements, hemodynamic measurements, breathing measurements, electric measurements, intracranial pressure measurements, etc. It should be appreciated that physiological sensor devices may be any type of medical sensor device.

In one embodiment, a physiological sensor that outputs digital signals may be connected to cable hub apparatus 160 through a smart or portable cable. Hereinafter a smart or portable cable refers to a cable that carries digital signals as well as stores data. A smart cable may store, for example, patient demographic data, physiology measurement data, and/or sensor life data (data relating to the remaining life of a sensor), etc. In one embodiment, the smart cable may transmit and store patient-related data and sensor-related data (e.g., physiological sensor data). Of course, connection via a smart cable is not required for digital sensor connectors 250.

The embodiment shown in FIGS. 2A and 2B also includes an analog sensor connector 255 adapted for a heart reference sensor (HRS) connection on the fourth side 245. An HRS may reduce use-errors related to movements of DPTs relative to patient's phlebostatic axis.

On a fifth side (back side, not shown) of external housing 161, a fastening mechanism for securing cable hub apparatus 160 to a bed rail or an IV pole may be provided. The fastening mechanism may be, for example, a pole clamp, and may be configurable such types of fastening mechanism are known to those of skill in the art.

It should be appreciated that the specific physical configuration of elements of cable hub apparatus 160 described herein is merely illustrative and merely provides examples and does not limit the invention. Clearly, adjustments may be made to the configuration within the spirit of the invention. For example, in a first alternative embodiment, push buttons 230 may be provided on first side 210 instead of second side 225; in a second alternative embodiment, the alarm silence button of push buttons 230 may be omitted; in a third alternative embodiment, LED indicators 220 may be omitted; and in a fourth alternative embodiment, analog sensor connector 255 adapted for HRS connection on fourth side 245 may be omitted. It should be appreciated that the various connectors, buttons, indicators may be included or omitted and may be on any side and/or position based upon design configurations.

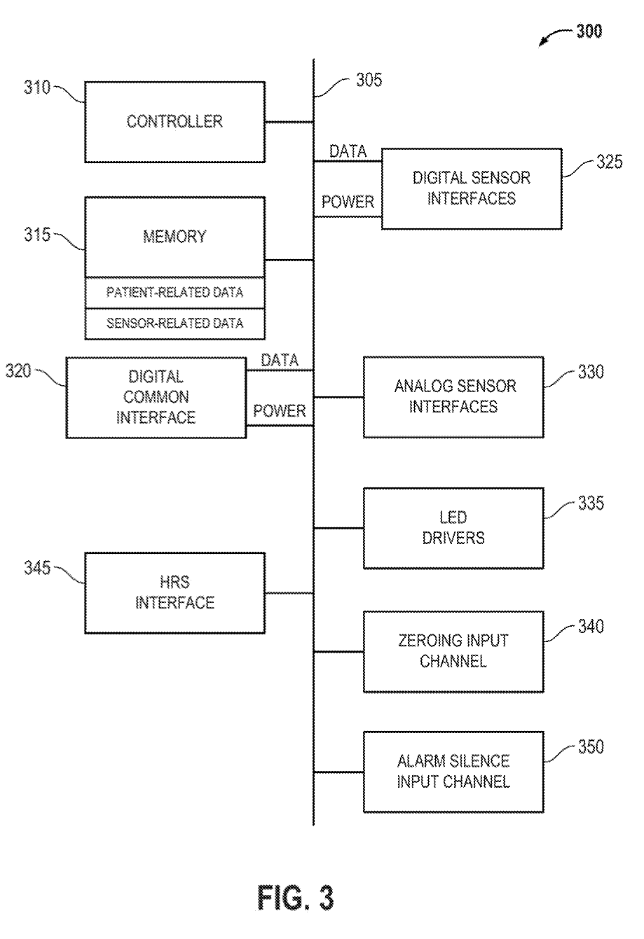

Referring to FIG. 3, a diagram illustrating an exemplary hardware architecture 300 for a cable hub apparatus 160 is shown. Hardware architecture 300 may reside inside external housing 161. One or more buses 305 may be provided to facilitate communications among the hardware components as well as to distribute power to the various components. A controller 310 may control and coordinate operations of various components of cable hub apparatus 160. A memory 315 may include a volatile portion and a non-volatile portion and may store operation codes for cable hub apparatus 160 as well as patient-related data and sensor-related data, as will be described in more detail below.

A digital common interface 320 may include sensor data lines, power supply, and in some embodiments, a heater signal supply (not shown). Through common interface 320, cable hub apparatus 160 may be connected to monitoring device 165 with a common interface cable 175, as previously described. As an example, the connection of sensor data lines of common interface 320 to controller 310 via buses 305 may be implemented with RS-485/UART (Universal Asynchronous Receiver/Transmitter) circuitry. The power supply may include, for example, +/−12V and +5V DC voltages.

In one embodiment, common interface 320 may also include circuitry that carries signals causing the start and/or reset of controller 310 and/or physiological sensors that output digital signals. A plurality of digital sensor interfaces 325 may be provided that correspond to digital sensor connectors 250 of FIGS. 2A and 2B. One or more digital physiological sensors that output digital signals may be connected to cable hub apparatus 160 through digital sensor interfaces 325 and via cables, such as smart cables, as previously described. The heater signal from common interface 320 may be routed to one of the plurality of digital sensor interfaces 320, to which a sensor including a heater that requires a heater signal may be connected. Data connections from controller 310 to digital sensor interfaces 325 via buses 305 may be implemented with RS-485/UART circuitry, and power in the form of DC voltages of +/−12V and +5/V may be provided to digital sensor interfaces 325 and analog sensor interfaces 330 which may pass the power on to connected sensors, via cables.

A plurality of analog sensor interfaces (e.g., DPT interfaces) 330 may be provided that correspond to analog sensor connectors 240 of FIGS. 2A and 2B. For example, one or more DPT sensors (or other types of analog physiological sensors may be connected via a cable to analog sensor interface 330). For example, connections from analog DPT interfaces 330 to controller 310 via buses 305 may be implemented with one or more analog-to-digital converters (ADCs) (not shown).

Additional circuitry may be provided to allow controller 310 to determine the connection/operation status of DPTs connected to analog DPT interfaces 330. For each of the plurality of analog DPT interfaces 330, a respective LED driver 335 that drives an LED indicator 220 of FIGS. 2A and 2B may be provided, and a respective zeroing input channel 340 that receives the signal generated by a depression of a zeroing push button of push buttons 230 may also be provided.

An HRS interface 345 may be provided that corresponds to analog sensor connector 255 adapted for an HRS connection of FIGS. 2A and 2B. Like those for DPT interfaces 330, the connection from HRS interface 345 to controller 310 via buses 305 may be implemented with an ADC. As described above, an HRS may reduce use-errors related to movements of DPTs relative to patient's phlebostatic axis. Moreover, an alarm silence input channel 350 may be provided to receive the signal generated by a depression of the alarm silence button of push buttons 230.

Of course, the description herein relating to FIG. 3 is illustrative and does not limit the invention. The hardware architecture 300 may be adjusted within the spirit of the invention. For example, when LED indicators 220 of FIGS. 2A and 2B are omitted, LED drivers 335 may also be omitted; when analog sensor connector 255 adapted for HRS connection is omitted, HRS interface 345 may also be omitted, etc.

As described above, memory 315 may store patient-related data and sensor-related data. The data may include, for example, a first use date of a sensor, a maximum age of a sensor, a unique patient ID, patient demographic data, patient physiological data, notes, and/or a transport flag, etc. Therefore, cable hub apparatus 160 may store patient demographic data, historical physiological data, sensor information, etc. in memory 310. The data stored in memory 310 may be retrieved, used, reused, or edited by a currently connected monitoring device 165. When a sensor is connected to cable hub apparatus 160 with a smart cable, which itself stores data, memory 315 may be utilized as a buffer, a backup, or a combination of both, for the data stored in the smart cable. The patient-related data and sensor-related data may be retrieved by a monitoring device 165 currently connected to cable hub apparatus through common interface cable 175. The data may be displayed on or otherwise utilized by monitoring device 165. Further, the patient-related data and sensor-related data stored in memory 315 may be edited through a monitoring device 165.

Therefore, according to embodiments of the invention, the cable hub circuitry includes: a plurality of analog sensor interfaces 330 that are configured to receive analog physiological sensor data from analog physiological sensors for the patient via a cable; and a plurality of digital sensor interfaces 325 configured to receive digital physiological sensor data from digital physiological sensors for the patient via a cable. Further, the controller 310 controls collecting the received physiological sensor data. For example, the controller 310 may package all of the received physiological sensor data into an appropriate digitized format and command the transmission through the digital common interface 320, which is configured to communicate the packaged data through the single common interface cable 175, to the patient monitoring device 165. Further, as previously described, the controller 310 may control the storing of received patient-related data and sensor-related data in memory 315 from the physiological sensor devices, as well as from the smart cable and/or the patient monitoring device 165. Moreover, the cable hub circuitry of the cable hub may be provided power via the single common interface cable 175 from the monitoring device 165. Further, in some embodiments, power may also be provided through the cable hub apparatus to the physiological sensors.

In this way, cable clutter may be reduced and transportation of patients simplified with the help of cable hub apparatus 160, according to embodiments of the invention, as described herein. When a patient is to be transported, common interface cable 175 may be disconnected from monitoring device 165 at the pre-transportation location. No separate handling of power supply cables is necessary because power is supplied through common interface cable 175 from the monitoring device 165. Cable hub apparatus 160 is easily transported together with the patient in a patient bed. When the patient is transported to the destination (e.g., from the ER to the ICU), common interface cable 175 is connected to monitoring device 165 at the post-transportation location to be powered. Patient- related data and sensor-related data stored in memory 315 is preserved during the transportation and is available for use and reuse once cable hub apparatus 160 is connected to monitoring device 165 at the post-transportation location. In other words, cable hub apparatus 160 may facilitate transfer of patient- and sensor-related data between one or more patient monitoring devices 165.

It should be appreciated that although cables have been described as connecting the physiological sensors to the cable hub apparatus and between the cable hub apparatus and the monitoring device, that in some embodiments, wireless connections may also be utilized.

It should be appreciated that aspects of the invention previously described may be implemented in conjunction with the execution of instructions by processors (e.g., controllers) of the devices, such as the cable hub apparatus and patient monitoring device, previously described. Processors may operate under the control of a program, routine, or the execution of instructions to execute methods or processes in accordance with embodiments of the invention. For example, such a program may be implemented in firmware or software (e.g. stored in memory and/or other locations) and may be implemented by processors and/or other circuitry of the devices previously described. Further, it should be appreciated that the terms processor, microprocessor, circuitry, controller, etc., refer to any type of logic or circuitry capable of executing logic, commands, instructions, software, firmware, functionality, etc.

The various illustrative logical blocks, modules, and circuits described in connection with the embodiments disclosed herein may be implemented or performed with a general purpose processor, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software/firmware module executed by a processor, or in a combination of the two. Memory to store data and modules may include RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A cable hub apparatus configured to communicate physiological data for a patient to a patient monitoring device comprising:
a plurality of analog sensor connectors configured to receive analog physiological sensor data for the patient;
a plurality of digital sensor connectors configured to receive digital physiological sensor data for the patient;
a controller to control collecting the received physiological sensor data; and
a digital interface configured to communicate through a single cable the received physiological sensor data to the patient monitoring device.

2. The cable hub apparatus of claim 1, wherein the single cable supplies power to the cable hub apparatus from the patient monitoring device.

3. The cable hub apparatus of claim 1, further comprising a memory to store patient-related data and sensor-related data to facilitate transferring the patient-related data and the sensor-related data between one or more patient monitoring devices.

4. The cable hub apparatus of claim 3, wherein at least one of the plurality of digital sensor connectors is connected to a sensor through a smart cable capable of storing patient-related data and sensor-related data.

5. The cable hub apparatus of claim 4, wherein the memory works as a buffer of the patient-related data and the sensor-related data stored in the smart cable.

6. The cable hub apparatus of claim 4, wherein the memory works as a backup of the patient-related data and the sensor-related data stored in the smart cable.

7. The cable hub apparatus of claim 1, further comprising a plurality of zeroing buttons for the plurality of analog sensor connectors in order to implement a zeroing function.

8. The cable hub apparatus of claim 1, wherein a housing of the cable hub apparatus is of approximately rectangular shape.

9. The cable hub apparatus of claim 1, wherein the plurality of analog sensor connectors are connectors for disposable pressure transducers (DPTs).

10. The cable hub apparatus of claim 9, further comprising:
a plurality of DPT holders; and
a configurable pole clamp.

11. The cable hub apparatus of claim 1, wherein the plurality of analog sensor connectors includes a plurality of connectors for disposable pressure transducers (DPTs) and a connector for a heart reference sensor (HRS).

12. A system configured to transmit physiological sensor data to a patient monitoring device, comprising:
one or more analog sensors;
one or more digital sensors; and
a cable hub apparatus comprising a plurality of analog sensor connectors configured to receive analog physiological sensor data from the analog sensors, a plurality of digital sensor connectors configured to receive digital physiological sensor data from the digital sensors, and a digital interface configured to communicate through a single cable the received physiological sensor data to the patient monitoring device.

13. The system of claim 12, wherein the single cable supplies power to the cable hub apparatus from the patient monitoring device.

14. The system of claim 12, wherein the cable hub apparatus further comprises a memory to store patient-related data and sensor-related data to facilitate transferring the patient-related data and the sensor-related data between one or more patient monitoring devices.

15. The system of claim 12, wherein at least one of the plurality of digital sensor connectors is connected to one of the digital sensors through a smart cable capable of storing patient-related data and sensor-related data.

16. The system of claim 12, wherein the cable hub apparatus further comprises a plurality of zeroing buttons for the plurality of analog sensor connectors in order to implement a zeroing function.

17. A method for transmitting physiological sensor data to a patient monitoring device, comprising:
receiving analog physiological sensor data at a plurality of analog sensor connectors of a cable hub apparatus;
receiving digital physiological sensor data at a plurality of digital sensor connectors of the cable hub apparatus;
converting the analog physiological sensor data into digitized analog physiological sensor data at the cable hub apparatus;
combining the digital physiological sensor data and the digitized analog physiological sensor data into combined digital data at the cable hub apparatus; and
transmitting the combined digital data to the patient monitoring device through a single cable from the cable hub apparatus.

18. The method of claim 17, wherein the single cable supplies power to the cable hub apparatus from the patient monitoring device.

19. The method of claim 17, further comprising storing at a memory of the cable hub apparatus patient-related data and sensor-related data to facilitate transferring the patient-related data and the sensor-related data between one or more patient monitoring devices.

20. The method of claim 17, further comprising performing a zeroing function for one of the plurality of analog sensor connectors at a zeroing button of the cable hub apparatus.
